SEQUENCE LISTING (6 sequences)

SEQ ID NO: 1 - protein, 21 aa, artificial sequence
  synthetic peptid
SPQAPRRPLPQVATLIDRFVK

SEQ ID NO: 2 - protein, 16 aa, artificial sequence
  synthetic peptide
TLDTLTAFYPGYLCSL

SEQ ID NO: 3 - protein, 20 aa, artificial sequence
  synthetic peptide
EVSGLSTERVRELAVALAQK

SEQ ID NO: 4 - protein, 33 aa, artificial sequence
  synthetic peptide
QLSNGNRTLTLFNVTRNDTASYKCETQNPVSAR

SEQ ID NO: 5 - protein, 9 aa, artificial sequence
  synthetic peptide
HLFGYSWYK

SEQ ID NO: 6 - protein, 24 aa, artificial sequence
  synthetic peptide
SSGCAFLSVKKQFEELTLGEFLKL

(19) United States
(12) Patent Application Publication
(10) Pub. No.: US 2013/0028915 A1
(43) Pub. Date: Jan. 31, 2013

(54) DENDRITIC CELL (DC)-VACCINE THERAPY FOR PANCREATIC CANCER

(71) Applicants: Anna Karolina Palucka, Dallas, TX (US); Jacques F. Banchereau, Montclair, NJ (US); Hideki Ueno, Plano, TX (US)

(72) Inventors: Anna Karolina Palucka, Dallas, TX (US); Jacques F. Banchereau, Montclair, NJ (US); Hideki Ueno, Plano, TX (US)

(73) Assignee: BAYLOR RESEARCH INSTITUTE, Dallas, TX (US)

(21) Appl. No.: 13/560,534

(22) Filed: Jul. 27, 2012

Related U.S. Application Data
(60) Provisional application No. 61/512,233, filed on Jul. 27, 2011.

(51) Int. Cl. A61K 39/00; A61P 35/00; C12N 5/0784; A61K 39/395

(52) U.S. Cl. 424/172.1; 424/185.1; 435/372

(57) ABSTRACT

Compositions and methods for eliciting therapeutic immunity and improving clinical outcomes in patients with pancreatic cancer are disclosed herein. The present invention describes a dendritic cell (DC)-vaccine comprising DCs pulsed with peptides derived from pancreatic cancer antigens for the therapy against pancreatic cancer. The vaccine described herein is safe, and leads to expansion of cancer specific T cells in patients with pancreatic cancer.

### CROSS-REFERENCE TO RELATED APPLICATIONS

This application claims priority to U.S. Provisional Application Ser. No. 61/512,233, filed Jul. 27, 2011, the entire contents of which are incorporated herein by reference.

### STATEMENT OF FEDERALLY FUNDED RESEARCH

This invention was made with U.S. Government support under Contract No. P50-AR05503 awarded by the National Institutes of Health (NIH). The government has certain rights in this invention.

### TECHNICAL FIELD OF THE INVENTION

The present invention relates in general to cancer therapy, and more particularly, to a dendritic cell (DC) vaccine pulsed with peptides derived from pancreatic cancer antigens for pancreatic cancer therapy.

### REFERENCE TO A SEQUENCE LISTING

The present application includes a Sequence Listing, and is hereby incorporated by reference in its entirety.

### BACKGROUND OF THE INVENTION

Without limiting the scope of the invention, its background is described in connection with cancer immunotherapy.

U.S. Pat. No. 6,805,869 issued to Guo (2004) provides a method for enhancing the immunogenicity of weakly immunogenic or non-immunogenic cells, resulting in a cellular vaccine that can stimulate T cell activation, which in turn leads to an effective immune response. The cellular vaccines of the present invention are useful for the prevention and treatment of diseases that develop and/or persist by escaping the immune response triggered by T cell activation. Such diseases include, for example, all cancers, natural and induced immune deficiency states, and diseases caused by infections with a variety of pathogens.

U.S. Patent Application Publication No. 2008020686 (Yu, 2008) provides a method of stimulating an immune response (e.g., to treat cancer) include administering to a patient a composition including dendritic cells that present cancer stem cell antigens. Compositions including cancer stem cell antigens are also provided herein. The cancer stem cell antigen composition in the Yu invention comprises one or more isolated peptides of CD133, CD90, CD44, CXCR4, Nestin, Musashi-1 (Msi1), maternal embryonic leucine zipper kinase (MELK), GLI1, PTCH1, Bmi-1, phosphoserine phosphatase (PSP), Snail, OCT4, BCRP1, MGMT, Bc1-2, FLIP, BCL-XL, XIAP, cIAP1, cIAP2, NAIP, or survivin.

U.S. Patent Application Publication No. 20090110702 (Wu et al. 2009) discloses the use of mesothelin as an immunotherapeutic target. Mesothelin induces a cytolytic T cell response. Portions of mesothelin that induce such responses are identified. Vaccines can be either polynucleotide- or polypeptide-based. Carriers for raising a cytolytic T cell response include bacteria and viruses. A mouse model for testing vaccines and other anti-tumor therapeutics and prophylactics comprises a strongly mesothelin-expressing, transformed peritoneal cell line.

### SUMMARY OF THE INVENTION

The present invention describes compositions and methods for the treatment of pancreatic cancer by the use of a dendritic cell (DC)-vaccine. The novel DC-vaccine of the present invention comprises DCs pulsed with peptides derived from pancreatic cancer antigens. The DC-vaccine of the present invention is safe, and leads to expansion of cancer specific T cells in a humans.

In one embodiment the instant invention provides an immunostimulatory composition for generating an immune response to a cancer, for prophylaxis, for therapy, or any combination thereof in a human subject comprising: one or more antigen loaded dendritic cells (DCs), wherein the DCs are granulocyte macrophage colony stimulating factor (GM-CSF) and interferon alpha 2b (IFN-α) stimulated DCs, wherein the antigens comprise: at least one mesothelin antigen, antigenic peptide, or a fragment thereof and at least one carcinoembryonic antigen (CEA), antigenic peptide, or a fragment thereof, wherein the one or more antigen loaded DCs are present in an amount sufficient to generate an immune response, for the prophylaxis, for the therapy or any combination thereof in the human subject.

In a related aspect the at least one mesothelin antigen is selected from at least one of SEQ ID NO: 1, SEQ ID NO: 2, SEQ ID NO: 3, or mesothelin peptides that can be presented by MHC class I and/or class II molecules and the at least one CEA antigen is selected from SEQ ID NO: 4, SEQ ID NO: 5, or CEA peptides that can be presented by MHC class I and/or class II molecules or any combinations thereof. In one aspect the composition may further comprise survivin. In another aspect the composition further comprises one or more TLR4 agonists, wherein the TLR4 agonists are selected from the group consisting of lipopolysaccharide (LPS), heat shock proteins (hsp), fibrinogen, heparan sulfate, hyaluronic acid, nickel, and any combinations thereof. In yet another aspect the composition further comprises one or more optional agents selected from the group consisting of an agonistic anti-CD40 antibody; an agonistic anti-CD40 antibody fragment; a CD40 ligand (CD40L) polypeptide; a CD40L polypeptide fragment; and any combinations thereof.

In a specific aspect the cancer is pancreatic cancer. The composition as described in the embodiment hereinabove is administered prior to, after, or concurrently with a chemotherapy regimen, a radiation therapy regimen, a surgical procedure, another immunotherapy regimen, or a monoclonal antibody treatment regimen. In another aspect the composition is administered subcutaneously or intravenously to generate one or more antigen-specific CD8⁺ T-cells in the human subject. In yet another aspect the DCs used in the composition hereinabove are autologous.

The present invention in another embodiment provides a method for making a dendritic cell (DC) vaccine for generating an immune response to a cancer comprising the steps of: i) isolating one or more monocytes from a human subject, wherein the monocytes comprise one or more DCs, ii) stimulating the one or more DCs by culturing the monocytes with granulocyte macrophage colony stimulating factor (GM-CSF) and interferon alpha 2b (IFN-a), and iii) loading the stimulated DCs with one or more antigens to make the immunostimulatory composition or the DC-vaccine, wherein the antigens comprise: a) at least one mesothelin antigen, antigenic peptide, or a fragment thereof and b) at least one carcinoembryonic antigen (CEA), antigenic peptide, or a fragment thereof.

In one aspect the method as described hereinabove further comprises the step of administering the DC-vaccine to the human subject to generate an immune response for prophylaxis, for therapy, or any combinations thereof. In another aspect of the method the at least one mesothelin antigen is selected from the group consisting of SEQ ID NO: 1, SEQ ID NO: 2, SEQ ID NO: 3, or any combinations thereof. In yet another aspect the at least one CEA antigen is selected from the group consisting of SEQ ID NO: 4, SEQ ID NO: 5, or any combinations thereof. In specific aspects of the method the monocytes are autologous and the cancer is a pancreatic cancer.

In yet another embodiment the present invention provides a method for prophylaxis, therapy, amelioration of symptoms or any combinations thereof against pancreatic cancer in a human subject comprising the steps of:

(i) identifying the human subject in need of prophylaxis, therapy, amelioration of symptoms or any combinations thereof against pancreatic cancer; and
(ii) administering a dendritic cell (DC)-vaccine to the human or subject, wherein the DC-vaccine comprises:
a) one or more antigen loaded dendritic cells (DCs), wherein the DCs are granulocyte macrophage colony stimulating factor (GM-CSF) and interferon alpha 2b (IFN-a) stimulated DCs, wherein the antigens comprise:
b) at least one mesothelin antigen, antigenic peptide, or a fragment thereof, wherein the mesothelin antigen is selected from the group consisting of SEQ ID NO: 1, SEQ ID NO: 2, SEQ ID NO: 3, or any combinations thereof; and
c) at least one carcinoembryonic antigen (CEA), antigenic peptide, or a fragment thereof, wherein the CEA antigen is selected from the group consisting of SEQ ID NO: 4, SEQ ID NO: 5, or any combinations thereof, wherein the one or more antigen loaded DCs are present in an amount sufficient to generate an immune response, for the prophylaxis, for the therapy or any combination thereof against pancreatic cancer in the human subject.

In one aspect of the method disclosed herein the vaccine may further comprises one or more of the following:

(i) survivin;
(ii) one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof; and
(iii) one or more agents selected from the group consisting of an agonistic anti-CD40 antibody; an agonistic anti-CD40 antibody fragment; a CD40 ligand (CD40L) polypeptide; a CD40L polypeptide fragment; and any combinations thereof.

In one aspect the vaccine disclosed herein is adapted for subcutaneous or intravenous administration to the human subject suffering from pancreatic cancer to generate one or more antigen-specific CD8⁺ T-cells in the human subject. In another aspect the vaccine is administered prior to, after, or concurrently with the chemotherapy regimen, the radiation therapy regimen, the surgical procedure, the immunotherapy regimen, or the monoclonal antibody treatment regimen.

A dendritic cell (DC)-vaccine composition for prophylaxis, for therapy, or any combination thereof against pancreatic cancer in a human subject is described in an embodiment of the present invention. The DC-vaccine as described comprises: one or more antigen loaded dendritic cells (DCs), wherein the DCs are granulocyte macrophage colony stimulating factor (GM-CSF) and interferon alpha 2b (IFN-a) stimulated DCs, wherein the antigens comprises: (i) at least one mesothelin antigen, antigenic peptide, or a fragment thereof, wherein the mesothelin antigen is selected at least one or SEQ ID NO: 1, SEQ ID NO: 2, or SEQ ID NO: 3, and (ii) at least one carcinoembryonic antigen (CEA), antigenic peptide, or a fragment thereof, wherein the CEA antigen is selected from at least one of SEQ ID NO: 4, SEQ ID NO: 5, wherein the one or more antigen loaded DCs are present in an amount sufficient to generate an immune response, for the prophylaxis, for the therapy or any combination thereof against pancreatic cancer in the human subject.

The DC-vaccine composition as described hereinabove further comprises: a) survivin, wherein the survivin comprises SEQ ID NO: 6, b) one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof, and c) one or more agents selected from the group consisting of an agonistic anti-CD40 antibody; an agonistic anti-CD40 antibody fragment; a CD40 ligand (CD40L) polypeptide; a CD40L polypeptide fragment; and any combinations thereof.

Another embodiment disclosed herein relates to a dendritic cell (DC)-vaccine composition for prophylaxis, for therapy, or any combination thereof against pancreatic cancer in a human subject comprising:

(i) one or more antigen loaded dendritic cells (DCs), wherein the DCs are granulocyte macrophage colony stimulating factor (GM-CSF) and interferon alpha 2b (IFN-α) stimulated DCs, wherein the antigens comprise: a) at least one mesothelin antigen, antigenic peptide, or a fragment thereof, wherein the mesothelin antigen is selected from the group consisting of SEQ ID NO: 1, SEQ ID NO: 2, SEQ ID NO: 3, or any combinations thereof and b) at least one carcinoembryonic antigen (CEA), antigenic peptide, or a fragment thereof, wherein the CEA antigen is selected from the group consisting of SEQ ID NO: 4, SEQ ID NO: 5, or any combinations thereof;
(ii) one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof; and
(iii) an optional pharmaceutically acceptable carrier, wherein the antigen loaded DCs and the TLR4 agonists are present in a sufficient amount such that the combination generates an immune response, for the prophylaxis, for the therapy or any combination thereof against pancreatic cancer in the human subject.

In one aspect the composition may optionally comprise survivin, wherein the survivin comprises SEQ ID NO: 6.

In yet another embodiment the present invention provides a method for prophylaxis, therapy, amelioration of symptoms or any combinations thereof against pancreatic cancer in a human subject comprising the steps of: (i) identifying the human subject in need of prophylaxis, therapy, amelioration of symptoms or any combinations thereof against pancreatic cancer and (ii) administering an autologous dendritic cell (DC)-vaccine to the human subject, wherein the DC-vaccine comprises: one or more antigen loaded dendritic cells (DCs), wherein the DCs are granulocyte macrophage colony stimulating factor (GM-CSF) and interferon alpha 2b (IFN-α) stimulated DCs, wherein the antigens comprise: a) at least one mesothelin antigen, antigenic peptide, or a fragment thereof, wherein the mesothelin antigen is selected from the group consisting of SEQ ID NO: 1, SEQ ID NO: 2, SEQ ID NO: 3, or any combinations thereof; b) at least one carcinoembryonic antigen (CEA), antigenic peptide, or a fragment thereof, wherein the CEA antigen is selected from the group consisting of SEQ ID NO: 4, SEQ ID NO: 5, or any combinations thereof, c) one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof, and d) an optional pharmaceutically acceptable carrier, wherein the antigen loaded DCs and the TLR4 agonists are present in a sufficient amount such that the combination generates an immune response, for the prophylaxis, for the therapy or any combination thereof against pancreatic cancer in the human subject.

The present invention further provides a method for promoting immunity for a prophylaxis, a therapy, amelioration of symptoms, or any combinations thereof against pancreatic cancer in a human subject comprising the steps of: (i) identifying the human subject in need of the prophylaxis, the therapy, amelioration of symptoms or any combinations thereof against the pancreatic cancer, (ii) isolating one or more autologous antigen presenting cells (APCs) from the human subject, wherein the APCs comprise macrophages, B cells, dendritic cells (DCs), or any combinations thereof, (iii) identifying one or more major histocompatibility complex (MHC) molecules present on a cell surface of the APCs isolated from the human subject, (iv) selecting two or more pancreatic cancer related antigens, antigenic peptides, or fragments thereof, wherein the selected antigens, antigenic peptides, or fragments thereof are matched with the one or more identified MHC molecules on the cell surface of the APCs, wherein the selected antigen comprises at least one mesothelin antigen and at least one carcinoembryonic antigen (CEA), (v) loading the isolated APCs with the selected antigens, antigenic peptides, or fragments thereof, and (vi) reintroducing the loaded APCs into the human subject for the promotion of immunity for the prophylaxis, the therapy, amelioration of symptoms, or any combinations thereof against the pancreatic cancer.

In one aspect of the method hereinabove the APCs comprise dendritic cells (DCs). In other specific aspects of the method hereinabove the at least one mesothelin antigen is selected from the group consisting of SEQ ID NO: 1, SEQ ID NO: 2, SEQ ID NO: 3, or any combinations thereof and the at least one CEA antigen is selected from the group consisting of SEQ ID NO: 4, SEQ ID NO: 5, or any combinations thereof.

The method as described hereinabove further comprises one or more optional steps, these steps include: i) loading the mesothelin and CEA antigen loaded APCs with survivin, ii) adding one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof, iii) adding one or more agents selected from the group consisting of an agonistic anti-CD40 antibody; an agonistic anti-CD40 antibody fragment; a CD40 ligand (CD40L) polypeptide; a CD40L polypeptide fragment; and any combinations thereof, and iv) dispersing the antigen loaded APCs with the optional agonists, the agents, or both in a pharmaceutically acceptable carrier. In yet another aspect of the method hereinabove the survivin comprises SEQ ID NO: 6. In another aspect of the method hereinabove the method may be used in a combination therapy with one or more strategies for the prophylaxis, the therapy, or both against pancreatic cancer, wherein the strategies are selected from the group consisting of chemotherapy; radiation therapy; surgery; immunotherapy; monoclonal antibody therapy; and any combinations thereof.

Another embodiment of the present invention relates to an immunostimulatory composition or a vaccine for generating an immune response against pancreatic cancer in a human subject cancer, for a prophylaxis, a therapy, or any combination thereof against the pancreatic cancer in a human subject comprising: at least one mesothelin antigen, antigenic peptide, or a fragment thereof, wherein the mesothelin antigen is selected from at least one of SEQ ID NO: 1, SEQ ID NO: 2, or SEQ ID NO: 3, at least one carcinoembryonic antigen (CEA), antigenic peptide, or a fragment thereof, wherein the CEA antigen is selected from at least one of SEQ ID NO: 4, SEQ ID NO: 5, or any combinations thereof, and one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof, wherein the at least one mesothelin antigen, the at least one CEA antigen, and the one or more TLR4 agonists are present in an amount sufficient to generate an immune response, for the prophylaxis, for the therapy or any combination thereof against pancreatic cancer in the human subject.

In another embodiment the present invention discloses an immunostimulatory composition or a vaccine for generating an immune response against pancreatic cancer in a human subject cancer, for a prophylaxis, a therapy, or any combination thereof against the pancreatic cancer in a human subject comprising: at least one mesothelin antigen, antigenic peptide, or a fragment thereof, wherein the mesothelin antigen is selected from at least one of SEQ ID NO: 1, SEQ ID NO: 2, or SEQ ID NO: 3 and at least one carcinoembryonic antigen (CEA), antigenic peptide, or a fragment thereof, wherein the CEA antigen is selected from at least one of SEQ ID NO: 4, SEQ ID NO: 5, or any combinations thereof, wherein the at least one mesothelin antigen and the at least one CEA antigen, are present in an amount sufficient to generate an immune response, for the prophylaxis, for the therapy or any combination thereof against pancreatic cancer in the human subject.

The composition as described hereinabove optionally comprises survivin, wherein the survivin comprises SEQ ID NO: 6, ii) one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof, and iii) one or more agents selected from the group consisting of an agonistic anti-CD40 antibody; an agonistic anti-CD40 antibody fragment; a CD40 ligand (CD40L) polypeptide; a CD40L polypeptide fragment; and any combinations thereof.

In yet another embodiment the present invention provides a method for prophylaxis, therapy, amelioration of symptoms or any combinations thereof against pancreatic cancer in a human subject comprising the steps of: i) identifying the human subject in need of prophylaxis, therapy, amelioration of symptoms or any combinations thereof against pancreatic cancer and ii) administering a therapeutically effective amount of an immunostimulatory composition or a vaccine to the human subject for the prophylaxis, the therapy, the amelioration of symptoms or any combinations thereof against pancreatic cancer, wherein the composition comprises: a) at least one mesothelin antigen, antigenic peptide, or a fragment thereof, wherein the mesothelin antigen is selected from at least one of SEQ ID NO: 1, SEQ ID NO: 2, or SEQ ID NO: 3, b) at least one carcinoembryonic antigen (CEA), antigenic peptide, or a fragment thereof, wherein the CEA antigen is selected from at least one of SEQ ID NO: 4, SEQ ID NO: 5, or any combinations thereof, and c) one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof.

In one aspect of the method hereinabove the composition may optionally comprise survivin, wherein the survivin comprises SEQ ID NO: 6. In another aspect of the method disclosed hereinabove the TLR4 agonist is LPS.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic showing the steps in the recall-memory assay and the analysis of the immune response pre and post-DC vaccination.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “Antigen Presenting Cells” (APC) refers to cells that are capable of activating T cells, and include, but are not limited to, certain macrophages, B cells and dendritic cells. “Dendritic cells” (DCs) refers to any member of a diverse population of morphologically similar cell types found in lymphoid or non-lymphoid tissues. These cells are characterized by their distinctive morphology, high levels of surface MHC-class II expression (Steinman, et al., Ann. Rev. Immunol. 9:271 (1991); incorporated herein by reference for its description of such cells). These cells can be isolated from a number of tissue sources, and conveniently, from peripheral blood, as described herein. Dendritic cell binding proteins refers to any protein for which receptors are expressed on a dendritic cell. Examples include GM-CSF, IL-1, TNF, IL-4, CD40L, CTLA4, CD28, and FLT-3 ligand.

For the purpose of the present invention, the term “vaccine” is intended to refer to a composition which can be administered to humans or to animals in order to induce an immune system response; this immune system response can result in a production of antibodies or simply in the activation of certain cells, in particular antigen-presenting cells, T lymphocytes and B lymphocytes. The vaccine composition can be a composition for prophylactic purposes or for therapeutic purposes, or both.

As used in this application, the term “amino acid” means one of the naturally occurring amino carboxylic acids of which proteins are comprised. The term “polypeptide” as described herein refers to a polymer of amino acid residues joined by peptide bonds, whether produced naturally or synthetically. Polypeptides of less than about 10 amino acid residues are commonly referred to as “peptides.” A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

As used herein, the term “antigen” refers to any antigen, which can be used in a vaccine, whether it involves a whole microorganism or a subunit, without regard to its specific configuration: peptide, protein, glycoprotein, polysaccharide, glycolipid, lipopeptide, etc. They may be viral antigens, bacterial antigens, or the like; the term “antigen” also comprises the polynucleotides, the sequences of which are chosen so as to encode the antigens whose expression by the individuals to which the polynucleotides are administered is desired, in the case of the immunization technique referred to as DNA immunization. They may also be a set of antigens, in particular in the case of a multivalent vaccine composition which comprises antigens capable of protecting against several diseases, and which is then generally referred to as a vaccine combination, or in the case of a composition which comprises several different antigens in order to protect against a single disease, as is the case for certain vaccines against whooping cough or the flu.

The term “mesothelin” as used herein refers to a mesothelin protein and fragments thereof which may be present on the surface of mammalian cells of a mammal such as rats, mice, and primates, particularly humans. The preferred nucleic acid and amino acid sequences of mesothelin are as described in PCT Published Application No. WO 97/25,068, U.S. patent application Ser. No. 08/776,271 and U.S. Provisional Application 60/010,166, all incorporated herein by reference. In addition, in Chang, K. & Pastan, I., Int. J Cancer 57:90 (1994); Chang, K. & Pastan, I., Proc. Nat'l Acad. Sci USA 93:136 (1996); Brinkmann U., et al., Int. J. Cancer 71:638 (1997); and Chowdhury, P. S., et al., Mol. Immunol. 34:9 (1997), each of which is incorporated herein by reference. “Mesothelin” also refers to mesothelin proteins or peptides which remain intracellular as well as secreted and/or isolated extracellular protein.

As used herein the term “carcinoembryonic antigen (CEA)” refers to a glycoprotein involved in cell adhesion. CEA is an oncofetal membrane glycoprotein, which provides a relevant tumor self-antigen target for the development of DNA vaccines for immunotherapy.

The term “antibodies” refers to immunoglobulins, whether natural or partially or wholly produced artificially, e.g. recombinant. An antibody may be monoclonal or polyclonal. The antibody may, in some cases, be a member of one, or a combination immunoglobulin classes, including: IgG, IgM, IgA, IgD, and IgE.

Antibodies against the proteins of the invention can be prepared by well-known methods using a purified protein according to the invention or a (synthetic) fragment derived therefrom as an antigen. Monoclonal antibodies can be prepared, for example, by the techniques as originally described in Kohler and Milstein, Nature 256 (1975), 495, and Galfre, Meth. Enzymol. 73 (1981), 3, which comprise the fusion of mouse myeloma cells to spleen cells derived from immunized mammals. The antibodies can be monoclonal antibodies, polyclonal antibodies or synthetic antibodies as well as fragments of antibodies, such as Fab, Fv or scFv fragments etc. As used herein, an antibody is said to “specifically bind” or “immunospecifically recognize” a cognate antigen if it reacts at a detectable level with the antigen, but does not react detectably with peptides containing an unrelated sequence, or a sequence of a different heme protein. Affinities of binding partners or antibodies can be readily determined using conventional techniques, for example, those described by Scatchard et al. (Ann. N.Y. Acad. Sci. USA 51:660 (1949)) or by surface plasmon resonance (BIAcore, Biosensor, Piscataway, N.J.). See, e.g., Wolff et al., Cancer Res. 53:2560-2565 (1993).

Furthermore, antibodies or fragments thereof to the aforementioned polypeptides can be obtained by using methods that are described, e.g., in Harlow and Lane “Antibodies, A Laboratory Manual”, CSH Press, Cold Spring Harbor, 1988. For example, surface plasmon resonance as employed in the BIAcore system can be used to increase the efficiency of phage antibodies that bind to an epitope of the protein of the invention (Schier, Human Antibodies Hybridomas 7 (1996), 97.varies.105; Malmborg, J. Immunol. Methods 183 (1995), 7-13). Antibodies, which bind specifically to a wild-type or a variant protein can be used for diagnosing or prognosing a related disorder, e.g., cancer.

The term “adjuvant” refers to a substance that enhances, augments or potentiates the host's immune response to a vaccine antigen.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

As used herein, the term “in vivo” refers to being inside the body. The term “in vitro” used as used in the present application is to be understood as indicating an operation carried out in a non-living system.

As used herein, the term “treatment” or “treating” refers to the administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes a novel dendritic cell (DC)-vaccine pulsed with peptides derived from pancreatic cancer antigens for therapy against pancreatic cancer. The vaccine described herein is safe, and leads to expansion of cancer specific T cells. A vaccination protocol for patients with pancreatic cancer using the DC-vaccine is also described. The novel DC vaccine of the present invention elicits a therapeutic immunity which might improve clinical outcomes in patients with pancreatic cancer who have an unmet medical need.

The novel DC-vaccine of the present invention comprises peptides derived from pancreatic cancer antigens to load DC vaccine. The candidate antigens include mesothelin carcinoembryonic antigen (CEA), survivin, and peptides thereof that can be presented by MHC class I and/or class II molecules, or combinations thereof. The DC was activated with LPS for generation of high avidity CD8⁺ T cell immunity. The inventors used immunogenicity data and those in the literature to design the peptides derived from the candidate antigens. The DCs in the present invention could also be activated in combination with a CD40 signal.

The present invention also describes studies carried out to assess the immunogenicity of DC vaccination in a patient with pancreatic cancer. Primary study endpoint was vaccine immunogenicity.

Pancreatic cancer is the 4^{th} leading cause of cancer related deaths in the US. Patients with pancreatic cancer have dismal survival and minimal benefit from current therapy. Thus, pancreatic cancer patients have an unmet medical need and, with minor exceptions, a dismal prognosis. Developing safe and well-tolerated therapeutic strategies providing disease control will thus have major impact. The present invention addresses this problem by developing an approach based on DC-vaccination. Immunotherapy can recruit tumor specific T cells and induce an oncolytic response thereby providing disease control with minimal adverse effects. Studies with adoptive T cell transfer demonstrate the capability of the immune system to deal with advanced tumors. The present inventors have developed a vaccination strategy that allows the induction and expansion of therapeutic T cells in vivo.

Cancer vaccines are in the renaissance era due to a number of Phase III clinical trials that show clinical benefit to the patients. For example, an active immunotherapy product Sipuleucel-T (APC8015) appears to contribute to prolonged median survival in patients with prostate cancer. This vaccine, known as Provenge® (Dendreon Corp., WA, USA) or Sipuleucel-T, comprises autologous, patient-derived DCs pulsed with a fusion protein consisting of the prostate tumor antigen prostatic acid phosphatase and GM-CSF. In a Phase III clinical trial, vaccination resulted in a 3-year survival advantage in vaccinated castration-resistant prostate cancer patients (31.7% survival) compared with placebo (23%).¹

Vaccines act through dendritic cells (DCs) that induce, regulate and maintain T cell immunity. Clinical studies conducted in patients with metastatic melanoma by the present inventors previously has demonstrated that a fraction of patients who received repeated vaccinations with melanoma-antigen loaded DCs obtained durable objective clinical responses and a long-term survival (over 5 years). In pancreatic cancer, vaccination with DC-vaccine pulsed with peptides derived from pancreatic cancer antigens is safe, and leads to expansion of T cells specific to pancreatic cancer antigens.

Immunotherapy is a novel therapeutic approach in pancreatic cancer that has the ability to recruit and activate tumor specific T-cells and induce an oncolytic response. Indeed, immunotherapy both active (vaccines) and passive (antibodies, T cells) is again on the front line of cancer treatment modalities. The work of the past decade clearly shows that antibodies can contribute to the control of tumors that express appropriate surface targets. T cells can reject established tumors when adoptively transferred into patients. Thus, the immune system can be harnessed for cancer therapy. However, passive immunotherapy might not lead to establishment and maintenance of memory T cells that might control tumor outgrowth on the long term. Active immunotherapy with vaccines has the potential to induce both tumor-specific effector and memory T cells. Vaccines act through dendritic cells (DCs), which induce, regulate and maintain T cell immunity. Previous studies using first generation DC vaccines pulsed with tumor antigens have shown that therapeutic immunity can be elicited. For example, an active immunotherapy product Sipuleucel-T (APC8015) appears to contribute to prolonged median survival in patients with prostate cancer. It is now clearly established that the goal of therapeutic vaccination is to generate antigen-specific CD8⁺ T cells, ideally in the presence of antigen-specific CD4⁺ T cells which are essential for establishment of long-lived memory.

The novel DC-vaccine of the present invention can be applied to other cancers by determining the MHC type of the patient and selecting T cell antigen epitopes that are presented by that MHC.

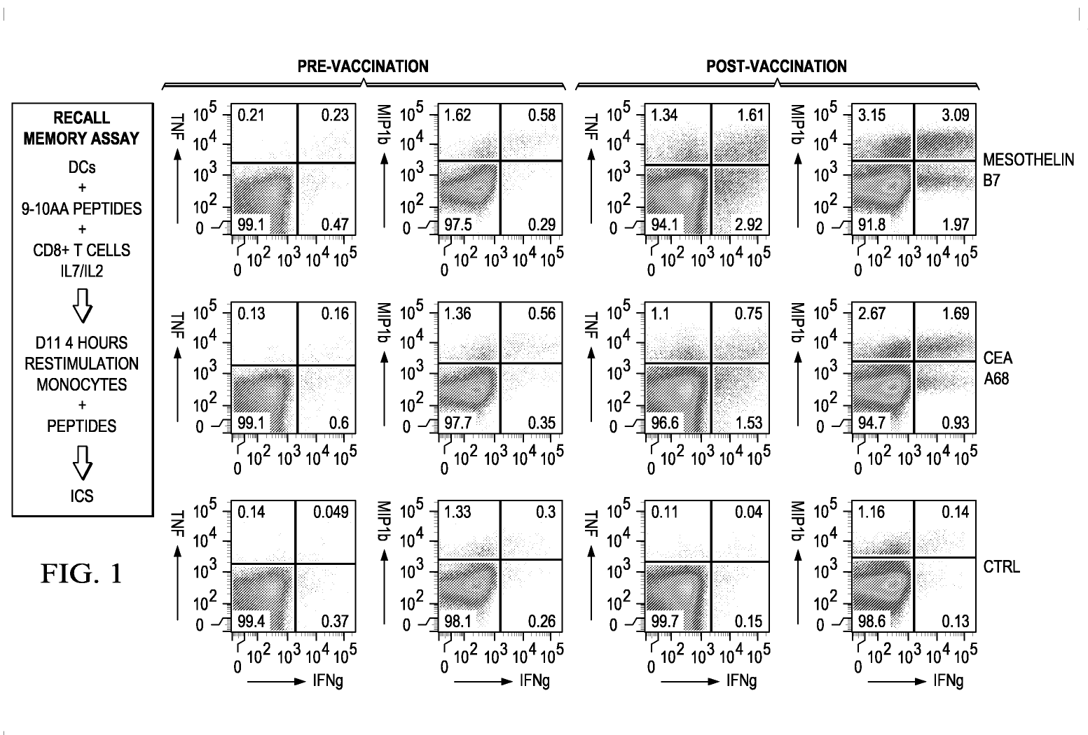

Using the novel DC-vaccine of the present invention, the inventors vaccinated, a patient with resected stage IV pancreatic cancer (ductal adenocarcinoma of the pancreas) who had residual disease treated with a standard protocol of Gemcitibine and 5FU. DC-vaccine was loaded with patient-specific synthetic peptides whose sequences were identified by the analysis of autologous tumor cells. The patient received repeated vaccinations, which were delivered one day after the last day of chemotherapy cycle. FIG. 1 illustrates the expansion of CD8⁺ T cells specific to pancreatic cancer antigens upon vaccination with the vaccine formulation described hereinabove.

Peptide Selection: The inventors selected peptides from Mesothelin, CEA, and Survivin, (Table 1). Other peptides that can be presented by MHC class I and/or class II molecules may also be used. For peptide design, the inventors analyzed a set of CD8+ T cell epitopes predicted by web-based software.² This software predicted peptide binders to more than 60 MHC class I molecules using Position Specific Scoring Matrices (PSSMs). The set of predicted CD8⁺ T cell epitopes was used to create a map to identify a region enriched with potential epitopes. Then, long peptides have been selected to contain 1) at least one published and validated epitope; and 2) several predicted epitopes. CEA61-69 has been identified as a CTL epitope for A3, but also was predicted to bind to other class I molecules, including A2, A11, and A24.

TABLE 1
Peptides for loading onto the DC-vaccines.

| | Position | Length | Sequence | Epitopes | Predicted |
|---|---|---|---|---|---|
| Mesothelin | 408-428 | 21 | SPQAPRRPLPQVATLIDRFVK (SEQ ID NO: 1) | B7 | A2, A11, |
| | | | | | B1 |
| Mesothelin | 437-452 | 16 | TLDTLTAFYPGYLCSL (SEQ ID NO: 2) | A1, A24 | A2 |
| Mesothelin | 73-92 | 20 | EVSGLSTERVRELAVALAQK (SEQ ID NO: 3) | A3, A68 | A2 |
| CEA | 192-224 | 32 | QLSNGNRTLTLFNVTRNDTASYKCETQNPVSAR | A68 | A2, A11 |
| | | | (SEQ ID NO: 4) | | |
| CEA | 61-69 | 9 | HLFGYSWYK (SEQ ID NO: 5) | A3 | A2, A11, |
| | | | | | A68 |
| Survivin | 81-104 | 24 | SSGCAFLSVKKQFEELTLGEFLKL | A2 | A3, A23, |
| | | | (SEQ ID NO: 6) | | B8 |

Vaccine Preparation: Vaccines were prepared in the cGMP Lab at BIIR from monocytes isolated from the apheresis by elutriation and cultured for four days with GM-CSF and IFN-α. Briefly, monocytes are positively selected from PBMCs and used to make DCs (current formulation of DC vaccine. DCs are loaded with a mixture of long peptides (1 μM at day 3 overnight) DCs are activated with LPS and with CD40L for the last 6 hrs of culture. Manufactured vaccines were stored in liquid nitrogen (vapor phase). The inventors have already demonstrated as described herein previously the feasibility and activity (both immune and clinical responses) of frozen IFN-DC vaccines in patients with stage 1V melanoma, in a patient with pancreatic cancer and in HIV patients. The endotoxin preparation (National Institutes of Health, Bethesda, Md.) that was used to activate the DC vaccine ex vivo is a reference endotoxin that has been certified by the FDA for in vivo use in healthy subjects.

The present invention describes a novel generation DC vaccine that elicits therapeutic immunity and improves clinical outcomes in patients with pancreatic cancer. The DC-vaccine of the present invention is optimized for generating tumor antigen-specific CD8+ T cell immunity in patients with pancreatic cancer. The principles of the novel therapeutic approach of the present invention can be applied to patients with other cancers.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 6,805,869: Cellular Vaccines and Immunotherapeutics and Methods for their Preparation.
U.S. Patent Publication No. 2008020686: Cancer Stem Cell Antigen Vaccines and Methods.
U.S. Patent Publication No. 20090110702: Mesothelin Vaccines and Model Systems and Control of Tumors.
¹. Dodson L F, Hawkins, W G, Goedgebuure P. Potential Targets for pancreatic cancer immunotherapeutics: Whole-Cell Vaccines. Immunotherapy. 2011; 3(4):517-537.
². RANKPEP. http://bio.dfci.harvard.edu/Tools/rankpep.html.

What is claimed is:

1. An immunostimulatory composition for generating an immune response to a cancer, for prophylaxis, for therapy, or any combination thereof in a human subject comprising:
one or more antigen-loaded dendritic cells (DCs) loaded with one or more antigens, wherein the DCs are granulocyte macrophage colony stimulating factor (GM-CSF) and interferon alpha 2b (IFN-α) stimulated DCs, wherein the one or more antigens comprise:
at least one mesothelin antigen or antigenic peptide; and
at least one carcinoembryonic antigen (CEA) or antigenic peptide, wherein the one or more antigen-loaded DCs are present in an amount sufficient to generate an immune response, for the prophylaxis, for the therapy or any combination thereof in the human subject.

2. The composition of claim 1, wherein the at least one mesothelin antigen is selected from at least one of SEQ ID NO: 1, SEQ ID NO: 2, SEQ ID NO: 3, mesothelin peptides that can be presented by MHC class I and/or class II molecules, or any combinations thereof.

3. The composition of claim 1, wherein the at least one CEA antigen is selected from SEQ ID NO: 4, SEQ ID NO: 5, CEA peptides that can be presented by MHC class I and/or class II molecules, or any combinations thereof.

4. The composition of claim 1, wherein the composition may further comprise survivin.

5. The composition of claim 4, wherein the survivin comprises SEQ ID NO: 6.

6. The composition of claim 1, wherein the composition further comprises one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof.

7. The composition of claim 1, wherein the composition further comprises one or more optional agents selected from the group consisting of an agonistic anti-CD40 antibody; an agonistic anti-CD40 antibody fragment; a CD40 ligand (CD40L) polypeptide; a CD40L polypeptide fragment; and any combinations thereof.

8. The composition of claim 1, wherein the cancer is pancreatic cancer.

9. The composition of claim 1, wherein the DCs are autologous.

10. A method for making a dendritic cell (DC)-vaccine for generating an immune response to a cancer comprising the steps of:
isolating one or more monocytes from a human subject, wherein the monocytes comprise one or more DCs;
stimulating the one or more DCs by culturing the monocytes with granulocyte macrophage colony stimulating factor (GM-CSF) and interferon alpha 2b (IFN-α); and
loading the stimulated DCs with one or more antigens, wherein the antigens comprise:
at least one mesothelin antigen, antigenic peptide, or a fragment thereof; and
at least one carcinoembryonic antigen (CEA), antigenic peptide, or a fragment thereof.

11. The method of claim 10, wherein the at least one mesothelin antigen is selected from the group consisting of SEQ ID NO: 1, SEQ ID NO: 2, SEQ ID NO: 3, mesothelin peptides that can be presented by MHC class I and/or class II molecules or any combinations thereof.

12. The method of claim 10, wherein the at least one CEA antigen is selected from the group consisting of SEQ ID NO: 4, SEQ ID NO: 5, CEA peptides that can be presented by MHC class I and/or class II molecules, or any combinations thereof.

13. The method of claim 10, wherein the monocytes are autologous.

14. The method of claim 10, wherein the cancer is a pancreatic cancer.

15. The method of claim 10, further comprising the optional steps of:
contacting the DCs with one or more one or more one or more TLR4 agonists, agents, or both, wherein the TLR4 agonists are selected from the group consisting of lipopolysaccharide (LPS); heat shock proteins (hsp); fibrinogen; heparan sulfate; hyaluronic acid; nickel; and any combinations thereof, wherein the agents are selected from the group consisting of an agonistic anti-CD40 antibody; an agonistic anti-CD40 antibody fragment; a CD40 ligand (CD40L) polypeptide; a CD40L polypeptide fragment; and any combinations thereof; and
loading the stimulated DCs with survivin.

16. A method for prophylaxis, therapy, amelioration of symptoms or any combinations thereof against pancreatic cancer in a human subject comprising the steps of:
identifying the human subject in need of prophylaxis, therapy, amelioration of symptoms or any combinations thereof against pancreatic cancer; and
administering a dendritic cell (DC)-vaccine to the human subject, wherein the DC-vaccine comprises:
one or more antigen loaded dendritic cells (DCs), wherein the DCs are granulocyte macrophage colony stimulating factor (GM-CSF) and interferon alpha 2b (IFN-α) stimulated DCs, wherein the antigens comprise:
at least one mesothelin antigen, antigenic peptide, or a fragment thereof, wherein the mesothelin antigen is selected from the group consisting of SEQ ID NO: 1, SEQ ID NO: 2, SEQ ID NO: 3, or any combinations thereof and
at least one carcinoembryonic antigen (CEA), antigenic peptide, or a fragment thereof, wherein the CEA antigen is selected from the group consisting of SEQ ID NO: 4, SEQ ID NO: 5, or any combinations thereof, wherein the one or more antigen loaded DCs are present in an amount sufficient to generate an immune response, for the prophylaxis, for the therapy or any combination thereof against pancreatic cancer in the human subject.

17. The method of claim 16, wherein the vaccine further comprises one or more of the following:
survivin;
one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof; and
one or more agents selected from the group consisting of an agonistic anti-CD40 antibody; an agonistic anti-CD40 antibody fragment; a CD40 ligand (CD40L) polypeptide; a CD40L polypeptide fragment; and any combinations thereof.

18. A method for promoting immunity for a prophylaxis, a therapy, amelioration of symptoms, or any combinations thereof against pancreatic cancer in a human subject comprising the steps of:
identifying the human subject in need of the prophylaxis, the therapy, amelioration of symptoms or any combinations thereof against the pancreatic cancer;
isolating one or more autologous antigen presenting cells (APCs) from the human subject, wherein the APCs comprise macrophages, B cells, dendritic cells (DCs), or any combinations thereof;
identifying one or more major histocompatibility complex (MHC) molecules present on a cell surface of the APCs isolated from the human subject;
selecting two or more pancreatic cancer related antigens, antigenic peptides, or fragments thereof, wherein the selected antigens, antigenic peptides, or fragments thereof are matched with the one or more identified MHC molecules on the cell surface of the APCs, wherein the selected antigen comprises at least one mesothelin antigen, at least one carcinoembryonic antigen (CEA), or at least one mesothelin peptide and at least one CEA peptide that can be presented by MHC class I and/or class II molecules;
loading the isolated APCs with the selected antigens, antigenic peptides, or fragments thereof; and
reintroducing the loaded APCs into the human subject for the promotion of immunity for the prophylaxis, the therapy, amelioration of symptoms, or any combinations thereof against the pancreatic cancer.

19. The method of claim 18, wherein the APCs comprise dendritic cells (DCs).

20. The method of claim 18, wherein the at least one mesothelin antigen is selected from the group consisting of SEQ ID NO: 1, SEQ ID NO: 2, SEQ ID NO: 3, or any combinations thereof.

21. The method of claim 18, wherein the at least one CEA antigen is selected from the group consisting of SEQ ID NO: 4, SEQ ID NO: 5, or any combinations thereof.

22. The method of claim 18, further comprising one or more optional steps:
loading the mesothelin and CEA antigen loaded APCs with survivin;
adding one or more TLR4 agonists, wherein the TLR4 agonists are selected from the group consisting of lipopolysaccharide (LPS); heat shock proteins (hsp); fibrinogen; heparan sulfate; hyaluronic acid; nickel; and any combinations thereof;
adding one or more agents selected from the group consisting of an agonistic anti-CD40 antibody; an agonistic anti-CD40 antibody fragment; a CD40 ligand (CD40L) polypeptide; a CD40L polypeptide fragment; and any combinations thereof; and
dispersing the antigen loaded APCs with the optional agonists, the agents, or both in a pharmaceutically acceptable carrier.
